(19) United States
(12) Patent Application Publication
(10) Pub. No.: US 2023/0355853 A1
(43) Pub. Date: Nov. 9, 2023

(54) AUTOMATIC REINFUSION OF BLOOD FOLLOWING A BLOOD TREATMENT THERAPY

(71) Applicant: B. Braun Avitum AG, Melsungen (DE)

(72) Inventors: Joana Sophie Schiller, Rheine (DE); Tobias Wuerschmidt, Hann. Muenden (DE)

(21) Appl. No.: 18/022,866

(22) PCT Filed: Aug. 24, 2021
(86) PCT No.: PCT/EP2021/073338

§ 371 Date: Feb. 23, 2023

(30) Foreign Application Priority Data
Sep. 2, 2020 (DE) 10 2020 122 936.4

(51) Int. Cl. A61M 1/36

(52) U.S. Cl. CPC A61M 1/361; A61M 1/3646; A61M 2230/207

(57) ABSTRACT

An extracorporeal blood treatment device includes an extracorporeal circuit, a dialyzer, a dialyzing liquid circuit and a control unit. A venous section and an arterial section of the extracorporeal circuit each includes a sensor configured to acquire a hematocrit percentage in a liquid flowing through the corresponding section. The control unit controls reinfusion of blood such that a dialyzing liquid is supplied from the dialyzing liquid circuit via a dialyzer membrane to the extracorporeal circuit, which displaces blood present in the extracorporeal circuit towards a patient to return the blood to the patient via the venous section and arterial section. Reinfusion in the corresponding section of the extracorporeal circuit is discontinued if it is acquired by the corresponding sensor or calculated or predicted by the control unit based on information acquired by the corresponding sensor that the hematocrit percentage falls below a predetermined limit value.

### CROSS-REFERENCE TO RELATED APPLICATIONS

This application is the United States national stage entry of International Application No. PCT/EP2021/073338, filed Aug. 24, 2021, and claims priority to German Application No. 10 2020 122 936.4, filed Sep. 2, 2020. The contents of International Application No. PCT/EP2021/073338 and German Application No. 10 2020 122 936.4 are incorporated by reference herein in their entireties.

### FIELD

The present disclosure relates to an extracorporeal blood treatment device, in particular dialysis machine, which is prepared or, respectively, configured for an automatic reinfusion/ refeeding of blood to a patient after a blood treatment therapy. Furthermore, the present disclosure relates to a method for automatically performing a reinfusion of blood after a blood treatment therapy.

### BACKGROUND

After a successful blood treatment therapy in which a blood of a patient has been purified extracorporeally using a dialyzer and a dialyzing liquid flowing through the dialyzer, generally the blood of the patient that is still in an extracorporeal circuit, for example in an arterial and venous tubing system (A/V tubing system), is returned to the patient. This process is referred to as reinfusion. Only when the blood still present in the extracorporeal circuit has been returned/ reinfused to the patient in a suitable manner is the patient completely disconnected from the extracorporeal tubing system. From the prior art a reinfusion method is known, for example, that uses an isotonic saline solution supplied from bags (hereinafter shortly referred to as saline bags). Thereby, after a performed blood treatment therapy, initially only the arterial end/ the arterial section of the extracorporeal circuit/ tubing system is disconnected from the patient and connected to a saline bag by a user/ nursing staff. Now, when a blood pump provided in the extracorporeal circuit rotates in therapy direction, the blood is displaced by the saline solution and flows through the arterial section, a dialyzer and a venous section of the extracorporeal circuit, and is returned to the patient via the venous end of the extracorporeal circuit.

Furthermore, from the prior art a reinfusion method via a substitute port or, respectively, connection is known. Thereby, after a performed blood treatment therapy, also initially only the arterial end/ the arterial section of the extracorporeal circuit/ tubing system is disconnected from the patient and connected to a substitute port or, respectively, connection of the extracorporeal blood treatment device by a user. In this case, when the blood pump rotates in therapy direction, the blood is displaced by the substitution liquid and flows through the arterial section, a dialyzer, and a venous section of the extracorporeal circuit, and is returned to the patient via the venous end of the extracorporeal circuit.

In both of the reinfusion methods mentioned, the reinfusion can be discontinued by stopping (of) the blood pump. A discontinuation of the reinfusion may, for example, be performed in a volume-controlled manner, e.g. when a predetermined volume has been reinfused. Furthermore, a discontinuation of the reinfusion may be performed in a time-controlled manner, for example when a predetermined reinfusion time has elapsed. Furthermore, in principle, a sensor may be provided also at the venous end/ in the venous section, which (sensor) is configured to detect a saline solution or a substitution liquid. For example, it is known from EP 1 996 253 B1 to discontinue the reinfusion by stopping the blood pump when a phase boundary between blood and saline solution has reached the venous end of the extracorporeal circuit.

In a disadvantageous way, the two mentioned reinfusion methods require either a substitute port to be provided at the dialysis machine or an external saline bag to be used. In other words, machines without an integrated substitute port must necessarily use a saline bag for the reinfusion, which is associated with high costs (for the saline bag). In addition, a user must actively intervene at least twice in the mentioned reinfusion methods. In particular, in these methods, the patient is initially arterially disconnected (action of the user), then the reinfusion takes place, and finally the patient is venously disconnected (action of the user).

In principle, it is desirable to keep interactions or, respectively, interventions by the user to a minimum. Further, it would be of advantage if the reinfusion can be performed without an external saline bag or, respectively, without a substitute port. In particular, it would be desirable to perform an automatic reinfusion of blood after a blood treatment therapy that requires no manual intervention by the user between the end of the blood treatment therapy and the start of the reinfusion.

Thereby, it is already known in principle from the prior art to control a reinfusion of blood automatically and immediately after the blood treatment therapy such that a dialyzing liquid is supplied from a dialyzing liquid circuit via a membrane of a dialyzer to the extracorporeal circuit, which during the reinfusion displaces the blood present in the extracorporeal circuit towards a patient in order to return the blood to the patient. In this context, reference is made only by way of example to EP 2 950 841 B1, EP 2 583 702 B1, EP 3 476 414 A1 or EP 1 684 825 B1. Thereby, from EP 2 950 841 B1 and EP 2 583 702 B1 a volume-controlled termination of the reinfusion is known. Thereby, in EP 2 583 702 B1, the volume-controlled terminating/ discontinuing of the reinfusion is started if a provided color sensor detects dialysis liquid. Furthermore, it is known from EP 3 476 414 A1 to automatically discontinue a reinfusion if a provided blood sensor acquires a blood concentration below a maximum allowed threshold value in an arterial section.

In principle, there is room for improvement in the prior art with regard to suitable termination criteria for discontinuing (of) the reinfusion. In particular, the prior art does not provide a suitable discontinuing (of) the reinfusion when blood is returned to the patient via both the venous section and the arterial section. Thereby, the prior art in particular does not take into account that during the reinfusion there is no phase boundary between blood and reinfusion liquid (dialyzing liquid), but that blood and reinfusion liquid/ dialyzing liquid mix. Furthermore, in the prior art an air entry into a patient during the reinfusion is not satisfactorily prevented.

Against this background, it is (an) object of the present disclosure to avoid or at least mitigate the aforementioned disadvantages from the prior art. In particular, an automatic initiation and implementation of a reinfusion after completion of a dialysis treatment is to be provided, which makes it possible for a user to need to intervene only after the reinfusion for a disconnecting of the patient. In other words, an interaction/ an intervention by the nursing staff should be kept to a minimum. Therefore, the reinfusion shall be performed in particular without external saline bag or, respectively, without substitute port. In particular, dialyzing liquid, that is supplied from the dialyzing liquid circuit via a membrane of a dialyzer to the extracorporeal circuit, shall be used as reinfusion liquid. It is also (an) object of the present disclosure to provide an improved control of a discontinuing of the reinfusion when blood is returned to the patient via both the arterial section and the venous section of the extracorporeal circuit, and to avoid an entry of air into the patient during the reinfusion.

### SUMMARY

The present disclosure provides an extracorporeal blood treatment device and a method for automatically performing a reinfusion of blood after a blood treatment therapy.

The disclosure first relates to an extracorporeal blood treatment device with: an extracorporeal circuit comprising an arterial section and a venous section, a dialyzer, and a dialyzing liquid circuit, wherein the extracorporeal circuit and the dialyzing liquid circuit are separated from each other via a membrane provided in the dialyzer; in the venous section a first sensor is provided, which is configured to acquire a hematocrit percentage in a liquid flowing through the venous section; in the arterial section a second sensor is provided, which is configured to acquire a hematocrit percentage in a liquid flowing through the arterial section; and the extracorporeal blood treatment device further comprises a control unit which is configured to control a reinfusion of blood (automatically/ (immediately) after the blood treatment therapy) such that a dialyzing liquid is supplied from the dialyzing liquid circuit via the membrane of the dialyzer to the extracorporeal circuit, which during/upon the reinfusion displaces the blood present in the extracorporeal circuit towards a patient to return the blood to the patient via both the venous section and the arterial section. The control unit is configured to discontinue the reinfusion via the venous section if/when it is acquired by the first sensor or calculated or predicted by the control unit on the basis of information acquired by the first sensor that the hematocrit percentage falls below a predetermined limit value, and to discontinue the reinfusion via the arterial section if/when it is acquired by the second sensor or calculated or predicted by the control unit on the basis of information acquired by the second sensor that the hematocrit percentage falls below the predetermined limit value.

In the dialyzing liquid circuit of the extracorporeal blood treatment device preferably both a dialyzer inlet valve and a dialyzer outlet valve are provided. The dialyzer inlet valve is preferably provided at a dialyzing liquid inflow upstream of the dialyzer. The dialyzer outlet valve is preferably provided at a dialyzing liquid outflow downstream of the dialyzer. Advantageously, a flow pump-inlet is arranged in the dialyzing liquid inflow (upstream of the dialyzer inlet valve). Further preferably, a flow pump-outlet is arranged in the dialyzing liquid outflow (downstream of the dialyzer outlet valve). The flow pump-inlet and/or the flow pump-outlet is/are preferably formed as gear pump(s).

According to the present disclosure, preferably after an end of the blood treatment therapy, the reinfusion is initiated by an excess pressure in the dialyzing liquid circuit or, respectively, on the dialyzing liquid side of the dialyzer. Due to the excess pressure, dialyzing liquid passes through the membrane of the dialyzer. This dialyzing liquid according to the disclosure serves as reinfusion liquid. The excess pressure on the dialyzing liquid side is preferably built up by a suitable control/ feedback control by the control unit. In particular, the flow pump-inlet may be controlled such that it/this pumps dialyzing liquid into the dialyzer. Thereby, preferably the flow pump-outlet is stopped. When the dialyzer outlet valve is closed, a suitable pressure (transmembrane pressure) is exerted on the membrane of the dialyzer by the dialyzing liquid. This pressure should not exceed a maximum permissible transmembrane pressure of the dialyzer used. Furthermore, the pressure (transmembrane pressure) is preferably selected such that a transfer of the dialyzing liquid with a defined flow (flow rate/ volume flow) is achieved. In particular, the defined flow may be set to a flow between 50 ml/min to 200 ml/min, for example 100 ml/min, depending on the dialyzer used.

Preferably, in the arterial section of the extracorporeal circuit, an (arterial) blood pump (preferably formed as roller pump to supply a fluid/ a liquid by squeezing of a tube) is provided, which is configured for a change of direction of rotation, and the control unit controls, during the reinfusion via the arterial section, the arterial blood pump such that it/this rotates against a therapy direction. Alternatively, however, the blood pump could also be configured to change from an occluded state to a non-occluded state (for example, by retractable rollers). In this case, the blood pump would not necessarily need to be configured for a change of direction of rotation in order to perform the arterial reinfusion. In this case, the arterial reinfusion could thus also be performed without rotary actuation of the blood pump against the therapy direction.

In order to return the blood to the patient via both the arterial section and the venous section of the extracorporeal circuit, the arterial blood pump is however preferably operated at a constant supply rate for producing a constant flow against the therapy direction. That is, the arterial flow pump preferably supplies a liquid from the dialyzer towards the arterial end of the extracorporeal circuit.

If, for example, the supply rate of the arterial flow pump is set such that a flow is generated in the arterial section against the therapy direction which corresponds to half the flow passing over/transgressing across/via the dialyzer membrane (for example 50 ml/min if the defined flow across the dialyzer membrane is 100 ml/min), it is automatically achieved that the flow in the venous section in therapy direction towards the venous end corresponds to the arterial flow. Since the flow introduced into the venous section is the difference between the flow passing over across the dialyzer membrane and the flow supplied by the arterial blood pump. Finally, if/when the tube clamps present in the extracorporeal circuit (arterial tube clamp and venous tube clamp) are opened, the blood in both the arterial section and the venous section of the extracorporeal circuit can be displaced towards the patient by the dialyzing liquid passing over.

According to a preferred embodiment/ the embodiment described above, the control unit is thus configured to perform the reinfusion via the venous section and the reinfusion via the arterial section in parallel/ simultaneously/ at the same time.

According to a further or, respectively, alternative preferred embodiment, however, the control unit may also be configured to perform the reinfusion via the venous section and the reinfusion via the arterial section serially/ successively/ sequentially. In principle, first the reinfusion via the venous section, and subsequently the reinfusion via the arterial section may be performed. Alternatively, in principle, likewise first the reinfusion via the arterial section, and only subsequently the reinfusion via the venous section may be performed.

For example, a serial blood return or, respectively, reinfusion may be performed such that initially the blood from the venous blood tubing side/ from the venous section is returned to a patient, and only then the blood from the arterial blood tubing side/ from the arterial section is reinfused. Thereby, preferably (as with the parallel reinfusion), after an end of the blood treatment therapy the reinfusion may be initiated by an excess pressure on the dialyzing liquid side of the dialyzer. Due to this excess pressure, the dialyzing liquid passes through the membrane of the dialyzer and serves as reinfusion liquid. Now, when the arterial blood pump is stopped and the venous tubing clamp is opened, the blood in the venous tubing segment/ section is displaced towards the patient by the passing over dialyzing liquid. After a discontinuation of the venous reinfusion, the reinfusion direction changes in an advantageous manner. In particular, the venous tube clamp is then closed and the arterial tube clamp is opened (if it was previously closed) or, respectively, remains open. Now, in an advantageous way, the arterial blood pump starts to rotate against the therapy direction and thus returns the blood from the arterial section/ blood tubing system to the patient. In this case, the supply rate of the arterial blood pump may be adjusted to the flow/ the flow rate of the dialyzing liquid passing over through the membrane. By a discontinuation of the arterial reinfusion the serial reinfusion process is overall terminated.

Alternatively, a serial blood return or, respectively, reinfusion may also be performed such that initially the blood from the arterial blood tubing side/ from the arterial section is returned to a patient, and only then the blood from the venous blood tubing side/ from the venous section is reinfused. Thereby, preferably after an end of the blood treatment therapy, the reinfusion may be initiated by an excess pressure on the dialyzing liquid side of the dialyzer. Due to this excess pressure, the dialyzing liquid passes through the membrane of the dialyzer and serves as reinfusion liquid. Now, when the venous tube clamp is closed or, respectively, remains closed, the arterial tube clamp is opened or, respectively, remains open, and the arterial blood pump begins to rotate against the therapy direction, the blood is returned to the patient from the arterial section/ blood tubing system. The supply rate of the arterial blood pump may be adjusted to the flow/ the flow rate of the dialyzing liquid passing over through the membrane. After a discontinuation of the arterial reinfusion (for example, by stopping of the arterial blood pump/ closing of the arterial tube clamp), the reinfusion direction changes in an advantageous manner. In particular, the venous tube clamp is then opened, and the blood is displaced in the venous tube segment/ section towards the patient by the dialyzing liquid passing over. By a discontinuation of the venous reinfusion the serial reinfusion process is overall terminated.

In other words, the serial blood return “arterial-venous” takes place analogously to the serial blood return “venous-arterial”. The machine components are merely controlled such that initially the arterial section is emptied and only then the venous section is emptied.

It is of advantage if the control unit is configured to control the reinfusion depending on the dialyzer used.

In particular, the control unit may thereby be configured to classify the dialyzer used, depending on the ultrafiltration coefficient (KUF) thereof, as a high-flux dialyzer or a low-flux dialyzer, and to perform, on the basis of the classification, an appropriate control (of the flow pump-inlet), in particular a high-flux control or a low-flux control.

The general principle is that a membrane of low-flux dialyzers has small pores and that a membrane of high-flux dialyzers has large pores. It is therefore self-evident that a liquid can pass through a membrane of high-flux dialyzers more easily/ better than through a membrane of low-flux dialyzers. Against this background, in order to achieve a desired flow (flow rate/ volume flow), the pressure of low-flux dialyzers must inevitably be set higher than that of high-flux dialyzers.

In an advantageous manner, the control of the reinfusion is thus according to the disclosure configured to the dialyzer used.

Thereby, the ultrafiltration coefficient may already be known to the control unit, for example, if information about the dialyzer used (comprising the ultrafiltration coefficient) is read in by a reading device prior to the blood treatment therapy and transmitted to the control unit or, respectively, the information is manually entered by a user prior to the blood treatment therapy, or the control unit may determine the ultrafiltration coefficient during the blood treatment therapy. For example, a constant ultrafiltration flow QUF (for example, 70 ml/min) may be set during the blood treatment therapy and the transmembrane pressure (may be) determined after a short setting time (for example, 40 seconds). From this information, the ultrafiltration coefficient (KUF) can be calculated in a known manner.

Preferably, the high-flux control is performed if/when the ultrafiltration coefficient is greater than a predetermined value, which is, for example, 20 ml/(min*mmHg), and the low-flux control is performed if/when the ultrafiltration coefficient is less than the predetermined value. In an advantageous manner, thus according to the disclosure the ultrafiltration coefficient of the corresponding dialyzer may be determined also for dialyzers from third-party manufacturers, and a classification as high-flux dialyzer or as low-flux dialyzer may be made to be able to control the reinfusion as a function thereof.

Preferably, the control unit is configured to perform a (safety) air removal step after the blood treatment therapy and before the reinfusion, by means of which air or, respectively, an air bubble is removed from the arterial section.

In an advantageous manner, an arterial tube clamp and an/the arterial blood pump are provided in the arterial section, and a venous expansion chamber/ air trap is provided in the venous section, and the control unit performs the (safety) air removal step such that initially the arterial section is clamped off for a predetermined period of time (for example two seconds) via the arterial tube clamp, while the arterial blood pump continues to run in (the) therapy direction, whereby the thus resulting negative pressure entrains the air or, respectively, air bubble present in the arterial section and the air bubble present in the arterial section and transports it into the venous section, where it is eliminated in the venous expansion chamber/ air trap.

According to the disclosure, it has been found that during the blood treatment therapy/ the dialysis treatment, especially when the extracorporeal blood circuit and dialyzer are filled with blood, an air bubble is present at an inlet of the arterial blood pump. However, in principle, air or air bubbles may also be present at other sections/ locations in the arterial section of the extracorporeal circuit. During the arterial reinfusion, in particular when operating the arterial blood pump against the therapy direction, there is generally a risk that an air bubble located in the arterial section can no longer be removed from the extracorporeal circuit (for example, because no arterial expansion chamber/ air trap is provided for removing air bubbles) and can therefore enter into the patient. This must be avoided at all costs, as an air bolus is harmful to the patient.

If this method step for safety (safety air removal step) is performed by the control unit before the reinfusion, a proper arterial blood return can be ensured.

Advantageously, the predetermined limit value for the hematocrit percentage is less than or equal to 10%, preferably between 2% and 5%, particularly preferably at 3%. For example, according to the disclosure, the predetermined limit value is set at a hematocrit percentage of 5% or a hematocrit percentage of 3%, so that the predetermined limit value is fallen short of if the hematocrit percentage is less than or equal to 5%/ less than or equal to 3%.

In particular, it has been found according to the disclosure that if the predetermined limit value for the hematocrit percentage is set to a percentage value which lies in the said range, a discontinuation of the reinfusion is not performed too early, i.e. is not yet performed if there is still too much blood of the patient in the extracorporeal circuit. Thereby, the predetermined limit value should also preferably not be set to a too small hematocrit percentage/ percentage value, for example less than 2%, since in this case the patient is already supplied with an excessive amount of dialyzing liquid/ reinfusion liquid. According to the disclosure, it is particularly preferred if the predetermined limit value is set at 3%. When, according to the disclosure, a limit value of 3% is referred to, this is of course not to be construed as “exactly 3%” but rather as “approximately/ circa 3%”.

According to a preferred embodiment, the first sensor is a venous safety air detector with (an) integrated red detector, which is configured to detect air or air bubbles in the liquid flowing through the venous section and to acquire the hematocrit percentage of the liquid as termination criterion of the reinfusion via the venous section, and the second sensor is an arterial safety air detector with (an) integrated red detector, which is configured to detect air or air bubbles in the liquid flowing through the arterial section and to acquire the hematocrit percentage of the liquid as termination criterion of the reinfusion via the arterial section.

In other words, an embodiment is preferably characterized in that a safety air detector with (an) integrated red detector is arranged or, respectively, provided both on the arterial side/ in the arterial section and on the venous side/ in the venous section. Thus, preferably two safety air detectors with integrated red detectors are provided in the extracorporeal blood treatment device, which can reliably detect air or, respectively, air bubbles and also acquire a hematocrit limit value as termination criterion for the reinfusion.

Since, according to this embodiment, air or, respectively, air bubbles can be reliably detected both in the arterial section and in the venous section (by providing two safety air detectors), the (safety) air removal step described above does not necessarily have to be performed. Since if air or, respectively, air bubbles were to be present in the arterial section, these - insofar as they detach and are conveyed towards the patient - would also be acquired by the safety air detector provided, and the control unit could in such a case in principle discontinue the reinfusion by stopping (of) the arterial blood pump or by closing (of) the arterial tube clamp or (of) the venous tube clamp.

Since, according to this embodiment, both in the arterial section and in the venous section, a safety air detector with integrated red detector is located (so-called SADRDV sensor), - in particular when a mixture of blood and dialyzing liquid flows through the arterial section/ the venous section/ the corresponding tube segment - a red value/ a colorimetric value of the blood-dialyzing liquid mixture in the corresponding tube segment can be determined. From the acquired red value, a hematocrit percentage of the blood-dialyzing liquid mixture can be determined quite accurately with the safety air detector with integrated red detector used. This also applies if the hematocrit percentage is already very low. As soon as the red detectors of the SADRDV sensors acquire a hematocrit percentage below the predetermined limit value mentioned (for example, less than 3%), the reinfusion is terminated in the corresponding tubing system in which it was acquired that the predetermined limit value is fallen short of.

In principle, it can happen that the blood in one section of the extracorporeal circuit/ in one segment of the blood tubing system (e.g. in the arterial section) is returned faster than the blood in the other section of the extracorporeal circuit/ the other segment of the blood tubing system (e.g. in the venous section). In this case, the control unit is configured to maintain or reduce the supply rate/ the flow of the dialyzing liquid passing over/transgressing the membrane of the dialyzer, and now to pass it completely over the section of the extracorporeal circuit in that the reinfusion has not yet been discontinued.

If the blood reinfusion via the venous section of the extracorporeal circuit is discontinued earlier, the control unit closes, for example, the venous tube clamp and controls the arterial blood pump so that it/this is operated at a supply rate of the passing over dialyzing liquid against the therapy direction. The passing over dialyzing liquid/ reinfusion liquid therefore displaces the remaining blood into the arterial part of the blood tubing system until the SADRDV sensor provided there detects the set hematocrit limit value.

In case of an earlier discontinuation of the blood reinfusion via the arterial section of the extracorporeal circuit, the arterial blood pump is stopped and the arterial tube clamp is closed. Then the passing over reinfusion liquid/ dialyzing liquid displaces the remaining blood into the venous section of the extracorporeal circuit until the SADRDV sensor provided there detects the set hematocrit limit.

Thereby, the air detectors of the SADRDV sensors continuously monitor that no air entry into the patient occurs. If an air bubble is detected, the control unit discontinues the reinfusion method. In particular, the control unit then closes the arterial tube clamp and the venous tube clamp, stops the blood pump, and removes the excess pressure on the dialyzer liquid side of the dialyzer (for example, by stopping the flow pump-inlet and opening the dialyzer outlet valve).

The reinfusion method described above may be applied analogously if the reinfusion is performed serially rather than in parallel. If, for example, during the serial reinfusion blood is returned to the patient initially via the venous section of the extracorporeal circuit, the red detector of the SADRDV sensor acquires/ monitors the hematocrit percentage of the blood or, respectively, the blood-dialyzing liquid mixture. If the red detector thereby detects a hematocrit percentage below the predetermined limit value (for example, less than 3%), the reinfusion via the venous section is discontinued or, respectively, terminated. In this exemplary case, the reinfusion follows via the arterial section. The red detector of the SADRDV sensor provided in the arterial section acquires/ monitors the hematocrit percentage of the blood or, respectively, the blood-dialyzing liquid mixture in the arterial section. If the red detector thereby detects a hematocrit percentage below the predetermined limit value (e.g. less than 3%), also the reinfusion via the arterial section is discontinued or, respectively, terminated.

According to a further or, respectively, alternative preferred embodiment, the first sensor is a venous safety air detector with (an) integrated red detector, which is configured to detect air or air bubbles in the liquid flowing through the venous section and to acquire the hematocrit percentage of the liquid as termination criterion of the reinfusion via the venous section, and the second sensor is a hematocrit sensor (HTC sensor), which is configured to acquire information about the hematocrit percentage in the liquid during the reinfusion and to forward this information to the control unit.

In other words, in principle, in the arterial section of the extracorporeal blood circuit instead of the safety air detector with integrated red detector (SADRDV sensor), a hematocrit sensor or, respectively, HTC sensor may be used for acquiring (of) a hematocrit percentage. In particular, in many extracorporeal blood treatment devices/ dialysis machines, in the arterial section of the extracorporeal circuit a hematocrit sensor (HTC sensor) is already provided as standard. This embodiment thus has the advantage that no additional safety air detector needs to be integrated at the arterial side/ in the arterial section (hardware change), but that in many machines only a software change needs to be made.

Since an HTC sensor does not include an air detector, in this embodiment it must be suitably ensured that no air enters the patient. To avoid having to integrate an additional air detector into the arterial section of the extracorporeal circuit, the (safety) air removal step described above is indispensable or, respectively, mandatory in this embodiment.

In particular, it has according to the disclosure been found that the safety air removal step described above enables that an air detector can be omitted in the arterial section of the extracorporeal circuit, and thus, in an advantageous manner, in the arterial section also only an inexpensive hematocrit sensor already present in many dialysis machines may be used.

However, it is problematic at this point that only an air detector with integrated red detector (SADRDV sensor) can reliably measure a hematocrit percentage at low percentage values. Hematocrit sensors (HTC sensors) can reliably measure a hematocrit value/ percentage only in a range between 20% and 55%, with limitations in a range between 10% and 55%. However, since the predetermined threshold value is according to the disclosure less than 10%, the hematocrit sensor cannot reliably acquire when the predetermined threshold value of the present disclosure is fallen short of. Against this background, it is necessary - in particular if a hematocrit sensor is used - that the control unit calculates or predicts on the basis of information acquired by the hematocrit sensor that/ when the hematocrit percentage falls short of/below the predetermined limit value.

Preferably, therefore, the control unit is configured to make, on the basis of the information about the hematocrit percentage in the liquid, a calculation or a prediction as to when the hematocrit percentage of the liquid falls short of/below the predetermined limit value. In other words, the control unit preferably uses the information transmitted to it by the hematocrit sensor, in particular information about the hematocrit percentage in the percentage range in which it/this reliably measures, to calculate or, respectively, predict when it is to be expected that the hematocrit percentage will fall below the predetermined limit value, and discontinues the reinfusion process on the basis of this calculation/ prediction.

Further preferably, the control unit is configured to make the calculation or the prediction by means of a linear function when performing the reinfusion via the venous section and the reinfusion via the arterial section serially/ successively/ sequentially, or if/when a reinfusion flow in the arterial section or the venous section is large or, respectively, is greater than a predetermined threshold value (e.g., 75 ml/min).

Further preferably, the control unit is configured to make the calculation or the prediction by means of a Gaussian function (first order) when performing the reinfusion via the venous section and the reinfusion via the arterial section in parallel/ simultaneously/ at the same time, or if/when a reinfusion flow in the arterial section or the venous section is small or, respectively, is smaller than a predetermined threshold value (e.g., 75 ml/min).

In other words, the control unit is preferably configured to make the calculation or the prediction by means of a linear function if/when the reinfusion flow is above a predetermined limit value/ threshold value, and to make the calculation or the prediction by means of a Gaussian function if/when the reinfusion flow is below a predetermined limit value/ threshold value. Thus, a linear function is used for the prediction if/when the reinfusion flow is large/ high and thus there are fewer mixing events between blood and dialyzing liquid during the reinfusion, and a Gaussian function is used for the prediction if/when the reinfusion flow is small/ low and thus there are more mixing events between blood and dialyzing liquid during the reinfusion.

The control unit is thus configured, when a hematocrit sensor (HTC sensor) is used for measuring (of) the hematocrit percentage, to intelligently evaluate the measurement results of the hematocrit sensor.

In summary, the disclosure thus relates to an extracorporeal blood treatment device with: an extracorporeal circuit, a dialyzer, a dialyzing liquid circuit, and a control unit. In both a venous section and an arterial section of the extracorporeal circuit, a sensor is provided, which is configured to acquire a hematocrit percentage in a liquid flowing through the corresponding section. The control unit is configured to control a reinfusion of blood such that a dialyzing liquid is supplied from the dialyzing liquid circuit via a dialyzer membrane to the extracorporeal circuit, which displaces during the reinfusion the blood present in the extracorporeal circuit towards a patient to return the blood to the patient via the venous section and the arterial section. Thereby, the reinfusion in the corresponding section of the extracorporeal circuit is terminated under sensor control if/when it is acquired by the corresponding sensor or it is calculated or predicted by the control unit on the basis of information acquired by the corresponding sensor that the hematocrit percentage falls below a predetermined limit value.

The present disclosure altogether provides an automatic and direct initiation of the reinfusion after the dialysis treatment. The reinfusion according to the disclosure saves material costs, since no saline bag is used for the reinfusion. In addition, the reinfusion is applicable even in machines without substitute port. The disclosure enables that the nursing staff does not need to actively intervene between the end of the dialysis treatment and the start of the reinfusion. In particular, there is no need for an interaction with the nursing staff for disconnecting (of) a patient port. Furthermore, the disclosure provides an improved discontinuation of the reinfusion when the reinfusion is performed via both the arterial section and the venous section of the extracorporeal circuit. Furthermore, it is ensured that there is no air entry into the patient.

Further, the present disclosure relates to a method for automatically performing (of) a reinfusion of blood after a blood treatment therapy comprising the steps: supplying (of) a dialyzing liquid from a dialyzing liquid circuit via a membrane of a dialyzer to an extracorporeal circuit; displacing (of) the blood present in the extracorporeal circuit towards a patient; returning (of) the blood to the patient via both a venous section of the extracorporeal circuit and an arterial section of the extracorporeal circuit; discontinuing (of) the reinfusion via the venous section if/when it is acquired by a first sensor provided in the venous section or if/when, on the basis of information acquired by the first sensor, it is predicted or calculated that a hematocrit percentage of a liquid containing blood flowing through the venous section falls short of/below a predetermined limit value; and discontinuing (of) the reinfusion via the arterial section if/when it is acquired by a second sensor provided in the arterial section or if/when, on the basis of information acquired by the second sensor, it is predicted or calculated that a hematocrit percentage of a liquid containing blood flowing through the arterial section falls short of/below a predetermined limit value.

In order to be able to perform the method according to the disclosure, an antegrade puncture (in direction of flow) of the arterial needle is preferably performed in the patient, since during a retrograde puncture (against the direction of flow) of the arterial needle turbulence may occur in the vessel.

After the method according to the disclosure has been performed or, respectively, the reinfusion via both the arterial section and the venous section has been discontinued and the arterial tube clamp and the venous tube clamp have been closed, the patient can be disconnected both arterially and venously by a nursing staff. Advantageously, the patient-side connections are then connected or, respectively, short-circuited. In other words, preferably the end of the arterial section is connected to the end of the venous section. Advantageously, then the method which is described in EP 3 231 466 B1 is performed. In particular, EP 3 231 466 B1 provides a suitable method for emptying of the blood tubing system and the blood-side dialyzer after the patient has been disconnected.

### BRIEF DESCRIPTION OF THE DRAWINGS

The disclosure is further explained below with reference to figures.

FIG. 1 shows an extracorporeal blood treatment device according to a first preferred embodiment of the present disclosure, with reference to which the automatic reinfusion according to the disclosure is explained;

FIG. 2 shows an extracorporeal blood treatment device according to a second preferred embodiment of the present disclosure, with reference to which the automatic reinfusion according to the disclosure is explained;

FIG. 3 shows a view of an arterial blood pump before a safety air removal step according to the disclosure is performed;

FIG. 4 shows a view of the arterial blood pump after the safety air removal step according to the disclosure has been performed;

FIG. 5 shows a diagram showing the curve of a volume flow of air supplied to a patient during the reinfusion via the arterial section of the extracorporeal circuit over time after the safety air removal step according to the disclosure has been performed;

FIG. 6 shows a diagram which ought to illustrate a prediction of a hematocrit percentage by means of a linear function when the reinfusion via the arterial section and the venous section is performed serially;

FIG. 7 shows a diagram which ought to illustrate a prediction of a hematocrit percentage by means of a linear function when the reinfusion via the arterial section and the venous section is performed in parallel; and

FIG. 8 shows a diagram which ought to illustrate a prediction of a hematocrit percentage by means of a Gaussian function when the reinfusion via the arterial section and the venous section is performed in parallel.

### DETAILED DESCRIPTION

The figures are of a schematic nature and are intended solely for the understanding of the disclosure. Identical elements are provided with the same reference signs. The features of the individual embodiments may be interchanged unless this is explicitly described otherwise.

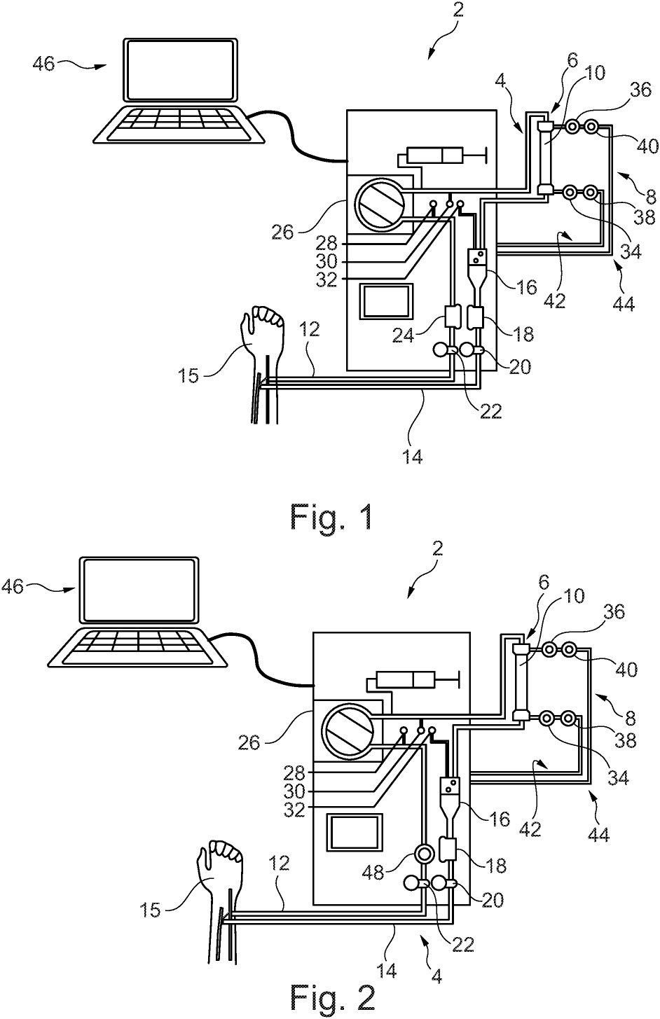

FIG. 1 shows an extracorporeal blood treatment device (dialysis machine) 2 according to a first preferred embodiment of the present disclosure, with reference to which the automatic reinfusion according to the disclosure is explained.

The extracorporeal blood treatment device 2 principally comprises an extracorporeal circuit (A/V tubing system) 4, a dialyzer 6, and a dialyzing liquid circuit 8. Thereby, the extracorporeal circuit 4 and the dialyzing liquid circuit 8 are separated from each other by a membrane 10 provided in the dialyzer 6.

The extracorporeal circuit 4 includes an arterial section 12, which is located upstream of the dialyzer 6, and a venous section 14, which is located downstream of the dialyzer 6.

As shown in FIG. 1, the arterial section 12 and the venous section 14 are coupled to a patient 15. In other words, an end of the arterial section 12 is coupled to an artery of the patient 15 and an end of the venous section 14 is coupled to a vein of the patient 15.

In the venous section 14 of the extracorporeal circuit 4, downstream of the dialyzer 6 (that is, starting from the dialyzer 6 in a direction towards the end of the venous section 14), a venous expansion chamber or, respectively, air trap 16, a venous safety air detector with (an) integrated red detector 18, and a venous tube clamp 20 are provided.

In the arterial section 12, starting from the patient-side end of the arterial section 12 in a direction towards the dialyzer 6, an arterial tube clamp 22, an arterial safety air detector with (an) integrated red detector 24 and an (arterial) blood pump 26 are provided. As can be seen in FIG. 1, the extracorporeal circuit 4 (in particular a blood pump adapter thereof) is already inserted into the blood pump 26, which is preferably formed as a roller pump or, respectively, peristaltic pump and is configured to supply a fluid/ a liquid by squeezing (of) a tube.

In the arterial section 12, an arterial pressure upstream of or, respectively, before the blood pump 26 may be measured by an arterial pressure sensor 28. Furthermore, a dialyzer inlet pressure downstream of or, respectively, after the blood pump 26 and upstream of or, respectively, before the dialyzer 6 (between the dialyzer 6 and the blood pump 26) may be measured via a dialyzer inlet pressure sensor 30. In the venous section 14, a venous pressure at/ downstream of the venous expansion chamber or, respectively, air trap 16 may be measured via a venous pressure sensor 32. The pressure sensors 28, 30, 32 provided in the extracorporeal circuit 4 may measure/ acquire/ monitor the pressure at the respective locations in the extracorporeal circuit 4 at which these are arranged/ provided.

The dialyzer liquid circuit 8 includes a dialyzer inlet valve 34, a dialyzer outlet valve 36, a flow pump-inlet 38 and a flow pump-outlet 40. However, it is generally sufficient if only one flow pump, for example the flow pump-inlet 38, is provided in the dialyzer liquid circuit 8. The dialyzer inlet valve 34 and the flow pump-inlet 38 are thereby provided/ arranged at a dialyzing liquid inflow 42 upstream of the dialyzer 6. The dialyzer outlet valve 36 and the flow pump-outlet 40 are provided/ arranged at a dialyzing liquid outflow 44 downstream of the dialyzer 6. The flow pump-inlet 38 and the flow pump-outlet 40 are preferably gear pumps.

The extracorporeal blood treatment device 2 further has a control unit 46, which is preferably formed as a processor, in particular as a central computing/ or, respectively, processing unit (CPU). The control unit 46 receives information from sensors which are provided in the extracorporeal blood treatment device 2. Merely by way of example, the sensors shown in FIG. 1 ought to be mentioned, i.e. the arterial pressure sensor 28, the dialyzer inlet pressure sensor 30, the venous pressure sensor 32, the arterial safety air detector with integrated red detector 24, the venous safety air detector with integrated red detector 18, etc. On the other hand, the control unit 46 controls or, respectively, actuates actuators which are provided in the extracorporeal blood treatment device 2. Merely by way of example, the valves, pumps, tube clamps, etc. shown in FIGS. 1, i.e., in particular the dialyzer inlet valve 34, the dialyzer outlet valve 36, the flow pump-inlet 38, the flow pump-outlet 40, the (arterial) blood pump 26, the arterial tube clamp 22, the venous tube clamp 20, etc., ought to be thereby mentioned.

The structure of an extracorporeal blood treatment device (dialysis machine) 2 (second preferred embodiment of the present disclosure) shown in FIG. 2 differs from the structure shown in FIG. 1 in terms of hardware only in that no arterial safety air detector with integrated red detector 24 is provided, but instead a hematocrit sensor (HTC sensor) 48 is arranged in the arterial section 12 of the extracorporeal circuit 4. Otherwise, the structure shown in FIG. 2 is identical to the structure shown in FIG. 1, so that the preceding explanations or, respectively, descriptions apply mutatis mutandis to the structure shown in FIG. 2 and are therefore not repeated.

FIG. 1 and FIG. 2 each show a state immediately after completion of a blood treatment therapy. In this state, it is desirable to return/ to reinfuse the blood still present in the extracorporeal circuit 4 to the patient.

In the embodiment shown in FIG. 1, a safety air removal step may optionally be performed after completion of the blood treatment therapy and before the start of the reinfusion. In the embodiment shown in FIG. 2, this safety air removal step is mandatory. The reason for this is that in the embodiment shown in FIG. 2, there is no safety air detector in the arterial section 12.

The control unit 46 performs the safety air removal step such that it initially clamps off the arterial section 12 for approximately two seconds via the arterial tube clamp 22 while the arterial blood pump 26 continues to run in therapy direction. Thereby a negative pressure is created in the arterial section 12, which entrains any air or air bubble 50 that may be present and carries it into the venous section 14, where the air or air bubble 50 is eliminated in the venous expansion chamber 16.

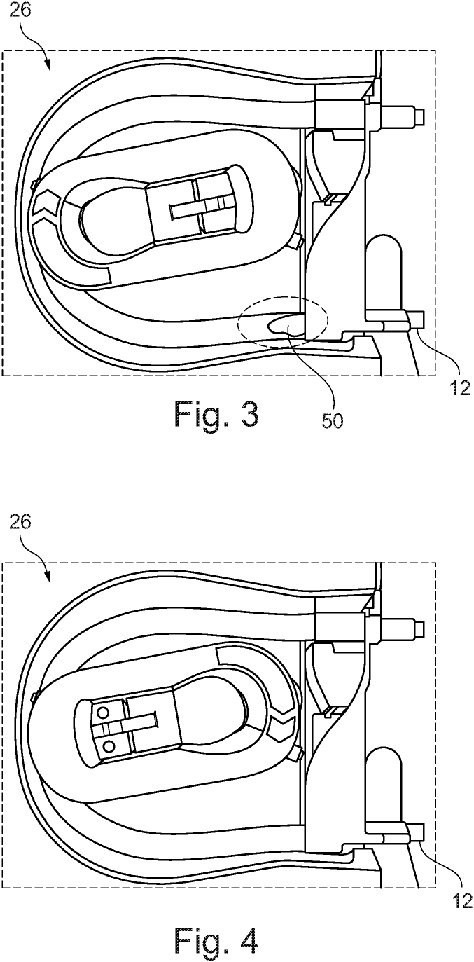

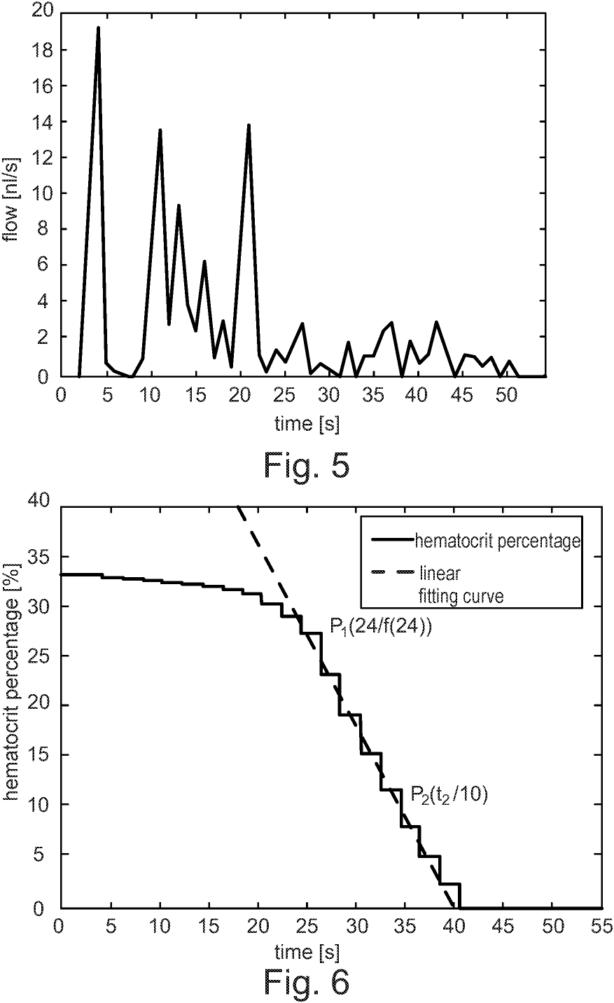

As can be seen from FIG. 3, after completion of a blood treatment therapy, often an air bubble 50 is present at an inlet of the arterial blood pump 26. Such an air bubble 50 can be suitably removed by the safety air removal step according to the disclosure. In particular, FIG. 4 shows the arterial blood pump 26 after the said safety air removal step has been performed. An air bubble 50 is no longer present in FIG. 4 and has been suitably eliminated by the safety air removal step. This is further illustrated by the diagram shown in FIG. 5. In FIG. 5, the volume/ the flow rate/ flow of air bubbles/ microbubbles over time during the arterial reinfusion is shown after the safety air removal step has been performed. It is shown that the volume/ the flow of the air bubbles is maximum 19 nl/s. This flow rate of air bubbles is far below a regimented maximum flow rate (500 nl/(s*kg)), so that the safety air removal step according to the disclosure enables a reliable removal of the air.

In the present case, the control unit 46 is principally configured to automatically control a reinfusion of blood after the blood treatment therapy or, respectively, if applicable, after the safety air removal step, such that a dialyzing liquid is supplied from the dialyzing liquid circuit 8 via the membrane 10 of the dialyzer 6 to the extracorporeal circuit 4, which during the reinfusion displaces the blood present in the extracorporeal circuit 4 towards the patient 15 in order to return the blood to the patient 15 both via the venous section 14 and via the arterial section 12.

The control unit 46 thereto actuates, according to the present disclosure, components present in the dialyzing liquid circuit 8 to create an excess pressure in the dialyzing liquid circuit 8 which causes that dialyzing liquid/ reinfusion liquid passes across the membrane 10 of the dialyzer 6. Preferably, the positive pressure is thereby created by the control unit 46 actuating the flow pump-inlet 38 to pump dialyzing liquid into the dialyzer 6 while the dialyzer inlet valve 34 is opened, the dialyzer outlet valve 36 is closed, and the flow pump-outlet 40 is stopped. Thus, a suitable pressure (transmembrane pressure) is exerted on the membrane 10 of the dialyzer 6 by the dialyzing liquid. This pressure causes that dialyzing liquid passes over from the dialyzing liquid circuit 8 to the extracorporeal circuit 4 across the membrane 10 of the dialyzer 6 at a defined flow (flow rate/ volume flow), for example 100 ml/min (depending on the dialyzer used).

In principle, the control unit 46 may perform the control depending on the dialyzer 6 used. For this purpose, the control unit 46 classifies the dialyzer 6 used, for example, as high-flux dialyzer or as low-flux dialyzer and performs, on the basis of this classification, in particular by accordingly actuating (of) the flow pump-inlet 38, a high-flux control or a low-flux control.

According to the present disclosure, the control unit 46 may perform the reinfusion via the arterial section 12 and via the venous section 14 in parallel or serially.

During the parallel reinfusion, the arterial blood pump 26 is operated by the control unit 46 against the therapy direction to supply blood/ liquid from the dialyzer 6 towards the end of the arterial section 12. For this purpose, the blood pump 26 may be set by the control unit 46 to a constant supply rate that is lower than the flow across the membrane 10 of the dialyzer 6. For example, the supply rate of the blood pump 26 may be set to 50 ml/min. When the defined flow across the membrane 10 of the dialyzer 6 is 100 ml/min, the flow in the venous section 14 in the therapy direction towards the end of the venous section 14 corresponds to the arterial flow. Now, when both the arterial tube clamp 22 and the venous tube clamp 20 are opened, the blood in both the arterial section 12 and the venous section 14 of the extracorporeal circuit 4 can be displaced towards the patient 15 by the dialyzing liquid passing over. The parallel reinfusion is terminated if/when both the arterial reinfusion and the venous reinfusion have been discontinued by the control unit 46 (by closing the arterial tube clamp 22 and the venous tube clamp 20).

If, during the parallel reinfusion, a blood reinfusion via the venous section 14 is discontinued earlier than a blood reinfusion via the arterial section 12, the control unit 46 closes the venous tube clamp 20 and increases the supply rate of the arterial blood pump 26. With an earlier discontinuation of the reinfusion via the arterial section 12, the control unit 46 stops the arterial blood pump 26 and closes the arterial tube clamp 22, so that the dialyzing liquid passing over now displaces only the remaining blood in the venous section 14.

In the serial reinfusion, for example, initially the blood from the venous section 14 may be returned to the patient 15, and only then the blood from the arterial section 12 be reinfused. In this case, the arterial blood pump 26 is stopped, the venous tube clamp 20 is opened, and the blood in the venous section 14 is displaced towards the patient 15 by the dialyzing liquid passing over. After a discontinuation of the venous reinfusion (by closing of the venous tube clamp 20), the arterial tube clamp 22 is opened (or remains open) and the arterial blood pump 26 begins to rotate against the therapy direction, so that the blood from the arterial section 12 is now also returned to the patient 15. The supply rate of the arterial blood pump 26 may thereby be adjusted to the flow of the dialyzing liquid passing over through the membrane 10. By a discontinuation of the arterial reinfusion, the serial reinfusion method is altogether terminated.

Alternatively, during the serial reinfusion, initially blood from the arterial section 12 may be returned to the patient 15, and only then the blood from the venous section 14 be reinfused. In this case, initially the venous tube clamp 20 is closed or, respectively, remains closed, the arterial tube clamp 22 is opened or, respectively, remains opened, and the arterial blood pump 26 starts to rotate against the therapy direction so that the blood from the arterial section 12 is returned to the patient 15. The supply rate of the arterial blood pump 26 is thereby adjusted to the flow of dialyzing liquid passing over through the membrane 10. After a termination of the arterial reinfusion (for example, by stopping of the arterial blood pump 26/ closing of the arterial tube clamp 22), the venous tube clamp 20 is opened, and the blood in the venous section 14 is displaced towards the patient 15 by the dialyzing liquid passing over. By a discontinuation of the venous reinfusion, the serial reinfusion method is altogether terminated.

In the following, the first preferred embodiment of the present disclosure according to FIG. 1 will be discussed in more detail. According to this embodiment, a safety air detector with integrated red detector is provided in both the arterial section 12 and the venous section 14, namely the arterial safety air detector with integrated red detector 24 and the venous safety air detector with integrated red detector 18. These two safety air detectors with integrated red detectors 18, 24 reliably detect air or air bubbles. If air or, respectively, air bubbles are detected, the control unit according to this embodiment discontinues the reinfusion by stopping (of) the arterial blood pump 26 or by closing (of) the arterial tube clamp 22 or (of) the venous tube clamp 20.

The arterial safety air detectors with integrated red detectors 18, 24 are configured to still determine a hematocrit percentage quite accurately even at relatively small percentage values. According to the first preferred embodiment, the arterial reinfusion is discontinued if/when the arterial safety air detector with integrated red detector 24 acquires that the hematocrit percentage in an end region of the arterial section 12 is below the predetermined limit value, which is preferably 3%. The venous reinfusion is discontinued if/when the venous safety air detector with integrated red detector 18 acquires that the hematocrit percentage in an end region of the venous section 12 is below the predetermined limit value.

In the following, the second preferred embodiment of the present disclosure according to FIG. 2 will be discussed in further detail. According to this embodiment, the venous safety air detector with integrated red detector 18 is provided in the venous section 14 and the hematocrit sensor/ HTC sensor 48 is provided in the arterial section 12.

Since the hematocrit sensor 48 can reliably measure a hematocrit value/percentage only in a range between 20% and 55%, the predetermined limit value is according to the disclosure less than 10%, in particular 3%, the measurement results of the hematocrit sensor 48 are according to the disclosure intelligently evaluated by the control unit 46 to be able to predict/ calculate when the predetermined limit value is fallen short of.

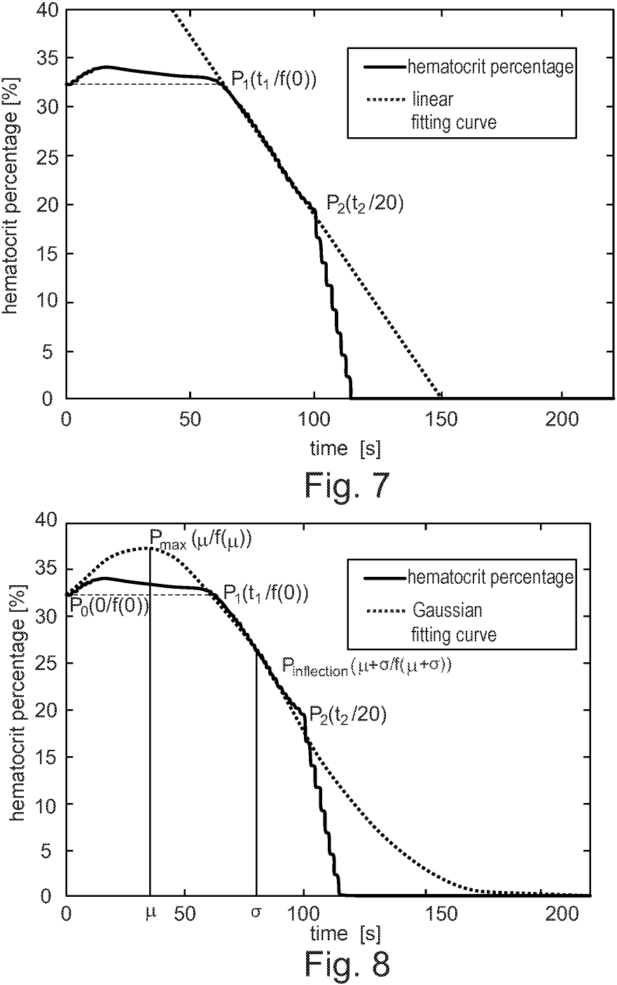

Such an intelligent evaluation is illustrated for the serial reinfusion (blood return via both the arterial section 12 and the venous section 14 at a flow rate of 100 ml/min) with reference to the diagram shown in FIG. 6. For the parallel reinfusion (blood return via both the arterial section 12 and the venous section 14 at a flow rate of 50 ml/min), such an intelligent evaluation is illustrated with reference to the diagrams in FIG. 7 and FIG. 8.

In order to be able to evaluate the hematocrit sensor 48 intelligently, a measurement curve of the hematocrit sensor 48 must be compared with an actual curve of the hematocrit percentage. For experimental purposes, an SADRDV sensor as reference sensor was integrated into the arterial section 12 for this purpose. For the experiments described below, a high-flux dialyzer available under the registered trademark XEVONTA® HI 23 was used.

In FIG. 6, for the serial blood reinfusion, only the curve of the hematocrit percentage for the arterial blood reinfusion measured by the hematocrit sensor 48 is shown. In the experiment, the blood tubing system between the hematocrit sensor 48 and the dialyzer 6 held approximately 40 ml. At a flow of 100 ml/min, the result is that the blood passes the hematocrit sensor 48 relatively undiluted for about 24 seconds (only slight decrease in the hematocrit percentage in FIG. 6). After 24 seconds, the hematocrit percentage decreases strongly until it reaches within 17 seconds a value of 0% (according to hematocrit sensor 48). When the hematocrit sensor 48 acquires the strong decrease of the hematocrit percentage, a point P1 can be defined. A second point P2 can be defined when the hematocrit percentage measured by the hematocrit sensor 48 is at a percentage value at which the hematocrit sensor 48 still measures accurately, such as at 20%. In FIGS. 6, P2 was defined at a hematocrit percentage of 10%. Through the points P1 and P, a linear function f(t) = -1.8 * t + 72.6 (for t > 24 s) is defined. For f(t) = 0, this results in a time of 40 seconds. However, due to the reference measurement with the SADRDV sensor, it is known that the hematocrit percentage after 55 seconds only falls below the predetermined limit value of 3%, resulting in an offset (for the dialyzer used, the flow set, and the tubing system used) of 15 seconds. Against this background, the control unit 46 may define the points P1 and P2 for the intelligent evaluation of the measurement results of the hematocrit sensor, define a linear function f(t) which passes through the points P1 and P2, and if according to the linear function f(t) = 0, add, if applicable, an offset to determine when the predetermined limit value is fallen short of. However, this approximation only works if the offset is small (in particular smaller than 20 seconds).

According to FIG. 7, an attempt was made to determine, also in the case of the parallel reinfusion, when the predetermined limit value is fallen short of, by fitting of a linear function to the measurement curve of the hematocrit sensor 48. As can be seen from FIG. 7, the curve of the hematocrit percentage measured by the hematocrit sensor 48 in the parallel reinfusion differs from that in the serial reinfusion. In particular, the hematocrit percentage in FIG. 7 does not decrease significantly during the first 63 seconds because the blood in the arterial section 12 of the extracorporeal circuit 4 passes the hematocrit sensor 48 relatively undiluted. Since there is a flow of only 50 ml/min, this process takes about twice as long as with the serial reinfusion. Thereafter, the hematocrit percentage drops with a constant gradient to 20% within 34 seconds. This is followed by a rapid decline to a hematocrit percentage of 0% within 18 seconds. Overall, thus, a different decrease in/of the hematocrit percentage is observed in the value range from 33% to 17% and in the value range from 17% to 0%. Against this background, it may be assumed that the reliable measuring range of the hematocrit sensor 48 extends to only about 20%. In this range, a linear function was fitted in FIG. 7, with the following functional equation: f(t) = -0.36*t + 54.23 [t>48 seconds]. For this function it is valid, that it has f(t) = 0 at 156 seconds. However, the actual time measured with the reference sensor until the predetermined limit value of 3% is fallen short of presently is 221 seconds. Against this background, in case of the parallel reinfusion, the offset is too large to predict a falling short of the predetermined limit value with a linear function.

As shown in FIG. 8, it has according to the disclosure been found that a first-order Gaussian function may be used, in the parallel reinfusion, for the prediction/ calculation, when the hematocrit percentage falls short of/below the predetermined limit value. In particular, the Gaussian function can be derived when the hematocrit percentage is recorded from the hematocrit sensor 48 up to 20%. In particular, three points P0, P1, and P2 of the hematocrit curve are used for the derivation of the Gaussian function, whereby P0 is the starting point, P1 is the point from which the hematocrit percentage decreases with a constant gradient, and P2 is the point up to which the hematocrit sensor measures reliably (approximately at 20%). Now, if the maximum Pmax of the Gaussian function is positioned such that it is exactly in the middle between P0 and P1 on the time axis, and the inflection point Pinflection of the Gaussian function is defined such that it is exactly in the middle between P1 and P2, the Gaussian function can be defined as shown in FIG. 8 as follows:

$$f \left(t\right) = 37 , 3 ∗ exp \left[- {\left(\frac{τ - 31 , 1}{57 , 1}\right)}^{2}\right]$$

As can be seen from FIG. 8, it is valid for this function that it has f(t) = 0 at about 216 seconds. Thus, with this function it can be predicted in a suitable way, in particular if a small offset is added, when the predetermined limit value of 3% will be/is fallen short of.

1. An extracorporeal blood treatment device comprising:
an extracorporeal circuit comprising an arterial section and a venous section;
a dialyzer; and
a dialyzing liquid circuit,
the extracorporeal circuit and the dialyzing liquid circuit being separated from each other by a membrane provided in the dialyzer;
a first sensor being provided in the venous section, the first sensor being configured to acquire a hematocrit percentage in liquid flowing through the venous section,
a second sensor being provided in the arterial section, the second sensor being configured to acquire a hematocrit percentage in liquid flowing through the arterial section, and
the extracorporeal blood treatment device further comprising a control unit configured to control a reinfusion of blood such that a dialyzing liquid is supplied from the dialyzing liquid circuit via the membrane to the extracorporeal circuit, which, during the reinfusion of blood, displaces blood present in the extracorporeal circuit towards a patient to return blood to the patient via both the venous section and the arterial section,
the control unit being configured to discontinue the reinfusion of blood via the venous section when the hematocrit percentage acquired by the first sensor or calculated or predicted by the control unit based on information acquired by the first sensor falls below a predetermined limit value,
the control unit further being configured to discontinue the reinfusion of blood via the arterial section when the hematocrit percentage acquired by the second sensor or calculated or predicted by the control unit based on information acquired by the second sensor falls below the predetermined limit value.

2. The extracorporeal blood treatment device according to claim 1, wherein the predetermined limit value is less than or equal to 10%.

3. The extracorporeal blood treatment device according to claim 1, further comprising an arterial blood pump in the arterial section,
the arterial blood pump being configured for a change of direction of rotation, and
the control unit controlling the arterial blood pump during the reinfusion of blood via the arterial section so that the arterial blood pump rotates against a therapy direction.

4. The extracorporeal blood treatment device according to claim 1, wherein the control unit is configured to perform the reinfusion of blood via the venous section and the reinfusion of blood via the arterial section in parallel.

5. The extracorporeal blood treatment device according to claim 1, wherein the control unit is configured to serially perform the reinfusion of blood via the venous section and the reinfusion of blood via the arterial section.

6. The extracorporeal blood treatment device according to claim 1, wherein the control unit is configured to control the reinfusion of blood as a function of the dialyzer used.

7. The extracorporeal blood treatment device according to claim 1, wherein the control unit is configured to perform, after a blood treatment therapy and before the reinfusion of blood, a safety air removal step, by which air or, respectively, an air bubble is removed from the arterial section.

8. The extracorporeal blood treatment device according to claim 7, further comprising an arterial tube clamp and an arterial blood pump provided in the arterial section, and a venous expansion chamber provided in the venous section,
wherein the control unit performs the safety air removal step such that initially the arterial section is clamped off for a predetermined period of time via the arterial tube clamp, while the arterial blood pump continues to run in a therapy direction, wherein a resulting negative pressure carries away the air or, respectively, air bubble present in the arterial section and conveys the air or, respectively, air bubble into the venous section, where the air or, respectively, air bubble is eliminated in the venous expansion chamber.

9. The extracorporeal blood treatment device according to claim 1,
wherein the first sensor is a venous safety air detector with integrated red detector, which is configured to detect air or air bubbles in the liquid flowing through the venous section and to acquire the hematocrit percentage of the liquid as termination criterion of the reinfusion of blood via the venous section, and
wherein the second sensor is an arterial safety air detector with integrated red detector, which is configured to detect air or air bubbles in the liquid flowing through the arterial section and to acquire the hematocrit percentage of the liquid as termination criterion of the reinfusion of blood via the arterial section.

10. The extracorporeal blood treatment device according to claim 7, wherein the first sensor is a venous safety air detector with integrated red detector, which is configured to detect air or air bubbles in the liquid flowing through the venous section and to acquire the hematocrit percentage of the liquid as termination criterion of the reinfusion via the venous section, and in that the second sensor is a hematocrit sensor which is configured to acquire information about the hematocrit percentage in the liquid during the reinfusion of blood and to forward said information to the control unit .

11. The extracorporeal blood treatment device according to claim 10, wherein the control unit is configured to make, based on the information about the hematocrit percentage in the liquid, a calculation or a prediction as to when the hematocrit percentage of the liquid falls below the predetermined limit value.

12. The extracorporeal blood treatment device according to claim 11, wherein the control unit is configured to make the calculation or the prediction by a linear function when performing the reinfusion of blood via the venous section and the reinfusion of blood via the arterial section serially or when a reinfusion flow in the arterial section or the venous section is large or, respectively, is greater than a predetermined threshold value.

13. The extracorporeal blood treatment device according to claim 11, wherein the control unit is configured to make the calculation or the prediction by a Gaussian function when performing the reinfusion of blood via the venous section and the reinfusion of blood via the arterial section in parallel or when a reinfusion flow in the arterial section or the venous section is small or, respectively, is smaller than a predetermined threshold value.

14. A method for automatically performing a reinfusion of blood after a blood treatment therapy, the method comprising the steps of:
supplying a dialyzing liquid from a dialyzing liquid circuit via a membrane of a dialyzer to an extracorporeal circuit;
displacing blood present in the extracorporeal circuit towards a patient;
returning the blood to the patient via both a venous section of the extracorporeal circuit and an arterial section of the extracorporeal circuit;
discontinuing the reinfusion of blood via the venous section when it is acquired by a first sensor provided in the venous section or if, based on information acquired by the first sensor, it is predicted or calculated that a hematocrit percentage of a liquid containing blood flowing through the venous section falls below a predetermined limit value; and
discontinuing of the reinfusion of blood via the arterial section if it is acquired by a second sensor provided in the arterial section or if, based on information acquired by the second sensor, it is predicted or calculated that a hematocrit percentage of a liquid containing blood flowing through the arterial section falls below a predetermined limit value.

15. The extracorporeal blood treatment device according to claim 1, wherein the predetermined limit value for the hematocrit percentage is between 1% and 5%.
